SEQUENCE LISTING (5 sequences)

SEQ ID NO: 1 - RNA, 20 nt, synthetic construct
  source 1..20: mol_type = other RNA
gccaagacctactacatgtc

SEQ ID NO: 2 - RNA, 20 nt, synthetic construct
  source 1..20: mol_type = other RNA
ggactgaccacctttaagcc

SEQ ID NO: 3 - DNA, 20 bp, unidentified
  source 1..20: mol_type = other DNA
gttcgtgaagctattaatca

SEQ ID NO: 4 - RNA, 36 nt, synthetic construct
  source 1..36: mol_type = other RNA
gttcgtgaagctattaatcagttttagagctatgct

SEQ ID NO: 5 - RNA, 36 nt, synthetic construct
  source 1..36: mol_type = other RNA
acctgggttattatcacgaagttttagagctatgct

(19) United States
(12) Patent Application Publication
(10) Pub. No.: US 2025/0312449 A1
(43) Pub. Date: Oct. 9, 2025

(54) METABOLIC REPROGRAMING OF ADOPTIVELY TRANSFERRED T CELLS TO POTENTIATE ANTITUMOR RESPONSE

(71) Applicant: H. LEE MOFFITT CANCER CENTER AND RESEARCH INSTITUTE INC., Tampa, FL (US)

(72) Inventors: Patrick HWU, Tampa, FL (US); Soraya Zorro MANRIQUE, Tampa, FL (US); Ana Lucia DOMINGUEZ, Tampa, FL (US); Paulo C. RODRIGUEZ, Tampa, FL (US); Yunfei WANG, Tampa, FL (US)

(21) Appl. No.: 18/867,896

(22) PCT Filed: Jun. 27, 2023
(86) PCT No.: PCT/US2023/069157

§ 371 Date: Nov. 21, 2024

Related U.S. Application Data
(60) Provisional application No. 63/367,234, filed on Jun. 29, 2022.

(51) Int. Cl. A61K 40/30; A61K 40/11; A61K 40/42; C12N 9/22; C12N 15/11; C12N 15/90

(52) U.S. Cl. CPC A61K 40/30; A61K 40/11; A61K 40/421; C12N 9/226; C12N 15/11; C12N 15/907; C12Y 102/04001; C12N 2310/20

(57) ABSTRACT

Disclosed herein are immune effector cells for use in adoptive cell transfer that have chemically- or genetically-inhibited PDHB (Pyruvate dehydrogenase E1 subunit beta) expression or activity. Also disclosed are methods of inhibiting or ablating PDHB expression in immune effector cells ex vivo and methods of using these cells to treat subjects with cancer. In some embodiments, the immune effector cells are further treated with a TIMS inhibitor or genetically engineered to ablate TIM3 expression. In some embodiments, the immune effector cells are further treated with a LAGS inhibitor or genetically engineered to ablate LAG3 expression. In some cases, the PDHB gene is disrupted by insertion of the gene encoding the chimeric receptor into the PDHB gene loci of the cell. Therefore, disclosed herein is a chimeric cell expressing a chimeric receptor.

### CROSS-REFERENCE TO RELATED APPLICATIONS

This application claims benefit of U.S. Provisional Application No. 63/367,234, filed Jun. 29, 2022, which is hereby incorporated herein by reference in its entirety.

### SEQUENCE LISTING

This application contains a sequence listing filed in ST.26 format entitled “320803_2890_Sequence_Listing” created on Jun. 27, 2023, and having 5,347 bytes. The content of the sequence listing is incorporated herein in its entirety.

### BACKGROUND OF THE INVENTION

Immunometabolism is an emerging field focused on the rewiring of metabolic pathways in immune cells and its impact on the immune response. The signaling of key metabolic pathways in T cells within the tumor microenvironment (TME), strongly impacts their activation, differentiation, survival, and antitumor response. Cancer cells compete with T cells for key nutrients such as glucose and amino acids; in addition, cancer cells produce metabolites that are toxic for T cells such as lactate and adenosine, inducing T-cell dysfunction. Therefore, the ex-vivo metabolic reprograming of adoptively transferred T-cells to confer adaptability to the harsh TME, is a very promising therapeutic strategy for advanced cancers.

### SUMMARY OF THE INVENTION

Disclosed herein are immune effector cells for use in adoptive cell transfer that have chemically- or genetically-inhibited PDHB expression or activity. Also disclosed are methods of inhibiting or ablating PDHB expression in immune effector cells ex vivo and methods of using these cells to treat subjects with cancer.

In some cases, the immune effector cells are treated ex vivo with an effective amount of a PDHB inhibitor to reduce or ablate PDHB expression or activity. In some embodiments, the PDHB inhibitor is an oligonucleotide, such as an antisense oligonucleotide, siRNA, or gRNA. In some cases the PDHB inhibitor is an aptamer or an antibody specific to the PDHB gene.

In some cases, the immune effector cells are genetically engineered ex vivo to inhibit or ablate PDHB expression. Methods for genetically manipulating gene expression and activity are known in the art and include gene editing and DNA recombination.

In some embodiments, the immune effector cells are further treated with a TIM3 inhibitor or genetically engineered to ablate TIM3 expression. In some embodiments, the immune effector cells are further treated with a LAG3 inhibitor or genetically engineered to ablate LAG3 expression.

In some embodiments, the immune effector cells are further treated with a checkpoint inhibitor, such as an anti-PD-1 antibody, anti-PD-L1 antibody, anti-CTLA-4 antibody, anti-OX40 antibody, or a combination thereof.

In some cases, the immune effector cells also express a chimeric receptor. In some embodiments, the chimeric receptor comprises a chimeric antigen receptor (CAR) polypeptide. CARs generally combine an antigen recognition domain with transmembrane signaling motifs involved in lymphocyte activation. The antigen recognition domain can be, for example, the single-chain variable fragments (scFv) of a monoclonal antibody (mAb) or a fragment of a natural ligand that binds a target receptor. CARs are generally made up of three domains: an ectodomain, a transmembrane domain, and an endodomain. The ectodomain comprises the antigen recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

In some cases, the PDHB gene is disrupted by insertion of the gene encoding the chimeric receptor into the PDHB gene loci of the cell. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous PDHB protein. Site-specific insertion of the transgene can be done, for example, by gene editing techniques, such as CRISPR or TALEN. In some cases, two different gene editing systems are used: one for integration of the transgene, and another one for effective ablation of all Sirt2 variants (for instance, with a target at exon 10, common to all of them).

Also disclosed are isolated nucleic acid sequences encoding the disclosed polypeptides, vectors comprising these isolated nucleic acids, and cells containing these vectors. The immune effector cells can be, for example, an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the lymphocytes are TILs.

Also disclosed is a method of providing an anti-cancer immunity in a subject, comprising administering to the subject an effective amount of an immune effector cell disclosed herein, thereby providing an anti-tumor immunity in the subject. Therefore, disclosed are methods of treating cancer in a subject that involves collecting immune effector cells from the subject, treating the lymphocytes ex vivo to inhibit PDHB expression, and transferring the modified lymphocytes back to the subject.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows Pmel CD8 T cells KO for PDHB or NTC adoptively transferred (ACT) into 7d-B16-tumor bearing mice. Tumors were isolated 7 days post ACT, and Pmel+ cells isolated (Thy-1.1+). Samples were stained for Live/Dead, CD45, CD3, CD8 and Thy-1.1+ to do flow cytometry analysis. FIG. 1A: Percentage of CD8 Thy1.1+ cell in the total tumor-infiltrating leukocyte population (CD45+). FIG. 1B. Percentage of Thy1.1+ cells (which tracks the adoptively transferred CD8 T cells) within the total CD8 population. The KO was performed by CRISPR/gRNA RNP transduction method (electroporation).

FIG. 2A shows 72 h-pre-activated and expanded Pmel splenocytes (48 h with aCD3+IL2+IL-7+IL-15, followed by 24 h expansion in IL-2) electroporated on d3 post isolation (purity level: ≥85% CD8 T cells) to introduce the RNP complex (gRNA construct+Cas9). Electroporated cells were left untouched for 48 h in 20% FBS+IL-2 (Cas9 required 24-48 h to excise the target gene). Within 24 h post electroporation, electroporation efficiency (%) was determined by flow cytometry, effectively transfected cells express ATTO_550 tracer. Knockout efficiency was evaluated by WB (KO vs NTC). The CD8 T cells KO or not for the GOI, are evaluated in in vivo and intro assays 4 d post electroporation to determine the impact of the PDHB disruption in T cells. FIG. 2B shows PDHB KO induction with different constructs: a) an IDT predesigned gRNA construct “AA”; b) a customized gRNA construct “AB”; c) a combination of “AA+AB”. KO levels were evaluated by WB; vinculin included in the assay as a housekeeping gene for loading control.

FIG. 3 shows PDHB KO CD8 T cells subjected to Mito Stress, Fatty acid β-oxidation (FAO) and Glycolysis Stress Tests (seahorse assays). The PDHB CD8 T cells displayed an impaired mitochondrial function with reduced respiratory capacity and FAO capacity with an increased glycolytic activity compared to NTC T cells.

FIGS. 4A to 4C show PDHB KO or NTC CD8 T cells adoptively transferred to 7d-B16-tumor bearing mice, and tumor sizes were recorded every 2-3 days. Some mice in addition to ACT, received aPD1. Data indicates that PDHB KO improve the effect of ACT vs NTC. The checkpoint blockade further enhances de the tumor growth control exerted by PDHB KO CD8 T cells. A. Average data, all conditions displayed, B. only ACT without combined therapy is depicted. C. Spider plots in which each line represent a mouse.

FIG. 5 shows PDHB KO or NTC CD8 T cells cocultured with target cells which express the human melanoma antigen gp100 (MC38/gp100) at different T cell: Cancer cell ration. After 3 h samples were stained for Caspase-3 and evaluated by flow cytometry. Killing is determined by the percentage of cancer cells which express Caspase-3.

FIG. 6 shows PDHB KO or NTC CD8 T cells activated with aCD3/aCD28-plate bound Abs+IL-2 and IFNγ expression was evaluated by flow cytometry. FMO plot indicate the cutting point for IFNγ expression. Based on Mean Fluorescent Intensity (MFI), cells were defined as hi or mid IFNγ-producing cells.

FIG. 7: 72 h-Pre-activated and expanded Pmel splenocytes (48 h with aCD3+IL2+IL-7+IL-15, followed by 24 h expansion in IL-2) were electroporated on d3 post isolation (purity level: ≥85% CD8 T cells) to introduce the RNP complex (gRNA construct+Cas9). Electroporated cells were left untouched for 48 h in 20% FBS+IL-2 (Cas9 required 24-48 h to excise the target gene). Within 24 h post electroporation, electroporation efficiency (%) was determined by flow cytometry, effectively transfected cells express ATTO_550 tracer. Knockout efficiency was evaluated by WB (KO vs NTC). The CD8 T cells KO or not for the GOI, were collected on d4 post electroporation and the transcriptome and metabolomics were performed.

FIG. 8 shows pathway analysis based on PDHB KO CD8 T cells transcriptome. Shown are two representative GSA analysis plots showing the upregulation of fatty acid pathways in the KO T cells.

FIG. 9. Plot displaying the most highly dysregulated metabolites; purines and pyrimidines were among the most significantly upregulated metabolites in the KO cells.

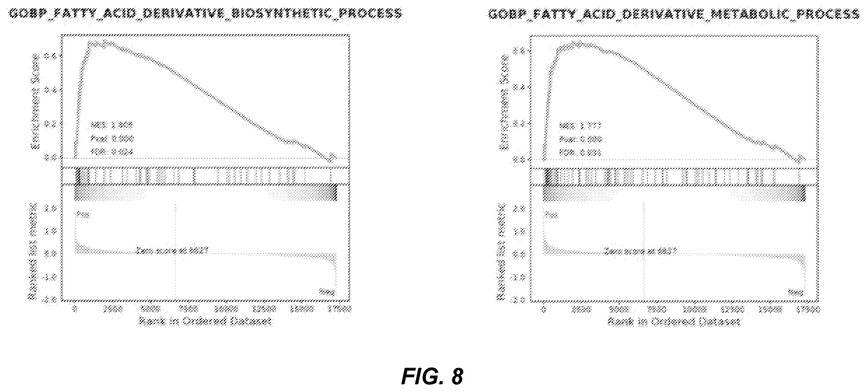

FIG. 10. Integrated transcriptome, metabolomics and pathway analyses. In red upregulated, in blue down regulated pathways in PDHB KO cells vs NTC. Upregulation of unsaturated fatty acid biosynthesis, glycolysis/gluconeogenesis, pyruvate metabolism, chemokine signaling and purine metabolism.

FIG. 11. Transcriptome analysis suggests that the PDHB KO CD8 T cells have an expression pattern of Tscm/Tcm cells. Heatmap indicating the genes related to the Tscm/Tcm profile. Volcano plot depicting the most significantly up (in red) and down (in blue) genes in PDHB KO cells (vs NTC). On the right sight, description of the function of some of the differentially expressed genes.

FIG. 12 a multiplex CRISPR/Cas9 RNP method to simultaneously target up to three genes. For the multiplex RNP targeting, each gene can be disrupted using an individual RPN complex that carries out a specific gRNA construct for the GOI. Thus, RPN 1 targets GOI 1, RNP 2 targets GOI 2, and RNP 3 targets GOI 3. IDT shared two fluorescent tracers not commercially available yet, to track each RNP complexes. The tracer that forms part of each RNP complex is tracked by the expression of either ATTO_488, ATT_647 or ATTO_550. The three surface molecules targeted by the multiplex RNP method: VISTA, PD-1 and LAG-3.

FIG. 13A is a flow chart indicating the sample preparation to perform KO. Different conditions were evaluated: single KO (single RNP for each of the target molecules VISTA, PD1 and LAG-3), double KO (combination of two different types of RNPs targeting either VISTA and PD1, or VISTA and LAG3), triple KO combination of all RNPs to simultaneously target VISTA, PD1 and LAG3 withing the same T-cell suspension. FIG. 13B shows electroporation efficiency determined within 24 h post electroporation by FC. Live T cells were gated and the presence of each of the constructs (RNPs) in CD8 T cells was tracked by the expression of ATTO_550, ATTO_488 and ATT_647. Each tracer reflects the presence of each type of RPN used to target different genes/molecules (VISTA, PD-1 and LAG-3).

FIG. 14 shows evaluation of KO levels (%) induced in CD8 T cells by each RPN construct targeting either VISTA (left plots), PD1 (middle plots) or LAG3 (right plots). Upper panel. Basal levels of marker expression in NTC T cells (% in black #s). Lower panel, the KO levels were determined by comparison to the expression of each maker in CD8 T cells transduced with NTC (percentage of KO efficacy in red, % KO; remaining protein expression in black % #). Flow cytometry done 4 days post electroporation. Live T cells were gated based on CD8 expression. This data indicates the maximum KO level that each RNP can achieve for the specific targets.

FIG. 15 shows evaluation of KO levels (%) induced in CD8 T cells when targeting two molecules simultaneously. Two RPNs constructs targeting either VISTA and PD1 (upper panel) or VISTA and LAG3 (lower panel) were utilized in the reaction. The KO levels were determined by comparison to the level of each maker in CD8 T cells transduced with NTC (basal levels indicated in previous slide, upper panel). The percentage of KO efficacy indicated in red, % KO; remaining protein expression in black % # (upper and lower panels). Flow cytometry done 4 days post electroporation. Live T cells were gated based on CD8 expression. This data indicates the maximum KO level that is achieved when two RNPs targeting two genes are used in a single reaction.

FIG. 16 shows evaluation of KO levels (%) induced in CD8 T cells when targeting three molecules simultaneously. Three RPNs constructs targeting VISTA, PD1 and LAG3 were utilized in the reaction. The KO levels were determined by comparison to the level of each maker in CD8 T cells transduced with NTC (basal values indicated in slide 18, upper panel). The percentage of KO efficacy indicated in red, % KO; remaining protein expression in black % #. Flow cytometry done 4 days post electroporation. Live T cells were gated based on CD8 expression. This data indicates the maximum KO level that is achieved when simultaneously using three RNPs to target three genes in a single reaction.

FIG. 17 illustrates the PDH complex.

FIG. 18 illustrates the role of the PHD complex in the metabolic pathway.

FIG. 19 illustrates a scheme used for T cell activation, expiation, CRISPR RNP transfection, PDHB KO evaluation, and validation.

FIGS. 20A to 2D show disruption of the Metabolic Hit PDHB Enhances Tumor Infiltration of Adoptively Transferred CD8 T Cells.

FIGS. 21A to 21C show mitochondrial stress test (FIG. 21), oxidative stress test (FIG. 21B), and glycolysis stress test (FIG. 21C) results for NTC and PDHB KO T cells.

FIGS. 22A and 20B show PDHB KO CD8 T cells display enhanced anti-tumor activity in Adoptive Cell Therapy (ACT) by intravenous (FIG. 22A) and intratumor (FIG. 22B) delivery.

FIGS. 23A and 23B show PDHB KO CD8 T cells display higher killing potential (FIG. 23A) and IFN-γ production (FIG. 23B).

FIG. 24 shows untargeted metabolomic profiling identified distinct metabolic reprograming in PDHB KO CD8 T cells.

FIG. 25 shows untargeted metabolomics revealed metabolic changes associated to the purine synthesis pathway. Boxes are upregulated metabolites in PDHB KO CD8 T cells.

FIG. 26 shows inosine hydrolysis is required for Increased IFN-γ production in PDHB KO CD8 T Cells.

FIG. 27 shows ¹³C6-Glucose tracer analysis in PDHB KO vs NTC CD8 T cells to elucidate the inosine source in PDHB KO CD8 T cells.

FIG. 28 shows glucose metabolism via the pentose phosphate pathway leads to increased inosine production in PDHB KO CD8 T cells.

FIG. 29 shows PDHB KO CD8 T cells express higher levels of fatty acids.

FIG. 30 shows integrated mRNA and metabolomics analysis.

FIG. 31 illustrates a potential model of PDH bypass via Acetyl-CoA synthetase (ACSS2), which is upregulated in PDHB KO T cells.

FIG. 32 shows PDHB disruption strongly increases CD8 T Cell proliferation. A B16.F10 spheroid pre-treated with IFNγ was cultured in a liquid-like-solid (LLS) material (packed granular gel) and treated with PDHB KO or WT T cells from D0-D3. Images from left-top, to left-bottom, and then right-top to right-bottom are t=0 h, t=5 h, t=10 h, t=15 h, t=20 h, t=25 h, t=30, h and t=35 h.

FIG. 33 shows hyperproliferative PDHB KO CD8 T Cells produce higher levels of IFN-γ.

FIG. 34 shows PDHB KO CD8 T cells contain a gene expression pattern of Tscm/Tcm cells. Tcf7 (TCF1): T cell proliferation, T cell stemness. CD7: CD7hi subset contains naive and memory cells. KLF2 (t.f.): T cell trafficking by promoting expression of the lipid binding receptor, S1P1, and the selectin, CD62L. KLF2 and S1Pr1: Aiding in memory T cell trafficking and retention in non-lymphoid tissue. Sema7A (Semaphorin 7A): negative regulation of T cell activation and function.

FIG. 35 shows a Western Blot of PBMCs from buffy coats of healthy human donor enriched for CD8 T cells electroporated with either NTC gRNA (Ctrl #1) or knocked out for PDHB (human constructs gRNA AA and AB). Band intensity of KO compared to NTC=21.43%.

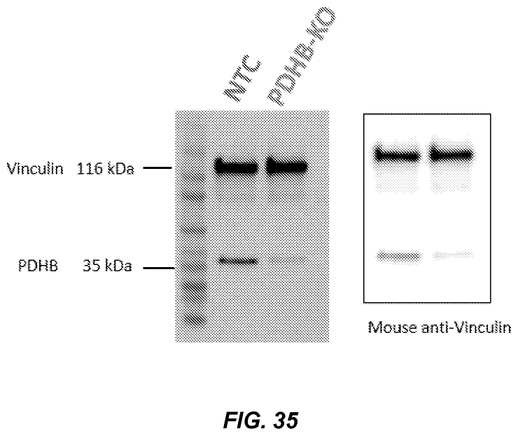

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Immune Effector Cells

The immune effector cells disclosed herein may be obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class Il molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### PDHB Inhibition or Ablation

In some embodiments, the immune effector cells are treated with a PDHB inhibitor or genetically engineered to ablate PDHB expression. For example, in some embodiments, the PDHB inhibitor is a gRNA that can be used with a Cas protein to silence human PDHB expression. For example, the gRNA can bind and target the nucleic acid sequence GCCAAGACCTACTACATGTC (SEQ ID NO:1) or GGACTGACCACCTTTAAGCC (SEQ ID NO:2).

In some embodiments, the immune effector cells are further treated with a TIM3 inhibitor or genetically engineered to ablate TIM3 expression.

In some embodiments, the immune effector cells are further treated with a LAG3 inhibitor or genetically engineered to ablate LAG3 expression.

As used herein the terms “inhibit” and “ablate” connote a partial or complete reduction in the expression and/or function of the PDHB polypeptide encoded by the endogenous gene. Thus, the expression or function of the PDHB gene product can be completely or partially disrupted or reduced (e.g., by 50%, 75%, 80%, 90%, 95% or more, e.g., 100%) in a selected group of cells (e.g., a tissue or organ) or in the entire animal.

Also disclosed are methods of disrupting PDHB expression in T cells ex vivo while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous PDHB protein.

In some embodiments, the transgene is inserted into the PDHB gene loci, thereby disrupting gene transcription. The transgene can be inserted at any loci within the PDHB gene that would disrupt gene transcription.

Site-specific insertion of the transgene can be done, for example, by gene editing techniques, such as CRISPR.

### Chimeric Antigen Receptors (CAR)

In some cases, the immune effector cells also expresses a chimeric receptor. In some embodiments, the chimeric receptor comprises a chimeric antigen receptor (CAR) polypeptide.

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak AD, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, Fc!RIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-Ia, LMP2, NCAM, p53, p53 mutant, Ras mutant, gpIOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvlll, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pI85erbB2,pI80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors and or oligonucleotides.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Therapeutic Methods

Immune effector cells disclosed herein can elicit an anti-tumor immune response against cancer cells. The anti-tumor immune response elicited by the disclosed immune effector cells may be an active or a passive immune response. In addition, the immune response may be part of an adoptive immunotherapy approach in which chimeric cells induce an immune response specific to the target antigen.

Adoptive transfer of immune effector cells is a promising anti-cancer therapeutic. Following the collection of a patient's immune effector cells, the cells may be genetically modified to ablate PDHB, then infused back into the patient.

The disclosed immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed immune effector cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed immune effector cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed immune effector cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed immune effector cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with cells for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: CRISPR/Cas9 RNP-Mediated Metabolic Reprograming of Adoptively Transferred T Cells to Potentiate Antitumor Response

To identify detrimental metabolic targets that could affect tumor T-cell infiltration, an in vivo shRNA screening was performed with a pooled library, covering ˜300 genes (a customized metabolome shRNA library) in a preclinical melanoma model, B16.F10.

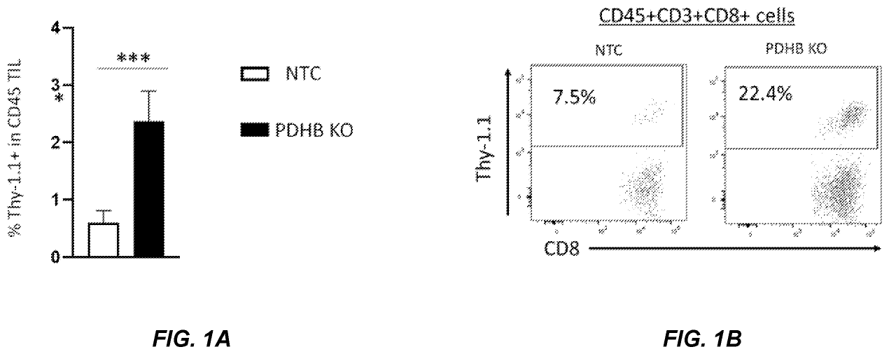

These studies revealed a set of metabolic candidate genes. The validation studies showed that the disruption of one of those candidates, Pdhb, in adoptively transferred T cells, enhances tumor infiltration (FIG. 1A, 1B).

Pdhb encodes for the E1 Subunit Beta of the Pyruvate Dehydrogenase (PDHB), an essential enzyme as it links glycolysis to the tricarboxylic acid (TCA) cycle two of the main sources of cellular energy.

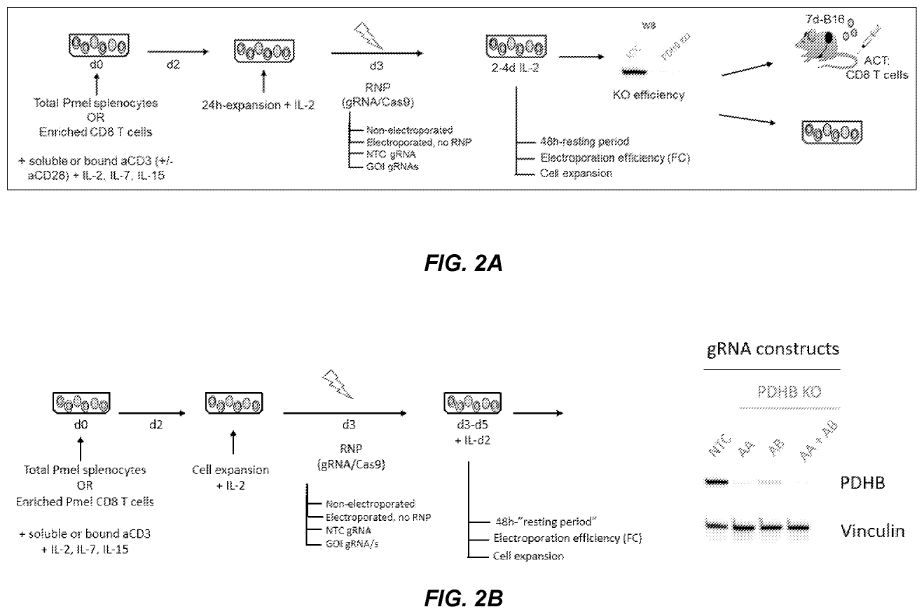

To achieve a highly efficient gene disruption, an improved CRISPR/Cas9 RNP transfection method was established, which allows to reach >90% knockout (KO) efficacy (FIG. 2A,2B). Through the CRISPR/Cas9 RNP method, mouse PDHB KO CD8 T cells were generated to evaluate PDHB function in in vivo and in vitro assays. The KO efficiency is evaluated by WB (FIG. 2,2B). FIG. 2B. To evaluate the best gRNA constructs to disrupt PDHB, different constructs were tested: a) the only available IDT predesigned gRNA construct “AA”; b) a customized gRNA construct designed by our group, which we named “AB”. The customized sequence was submitted to IDT for its generation (IDT named it “sequence 1”); c) combination of “AA”+“AB” (which we called “combo” or “AA+AB”). As targeting>1 DNA region may reduce the risk of getting DNA rearrangement, it was predicted that the “combo” option might be the best strategy to KO PDHB in CD8 T cells. In fact, we observed a slightly improved effect in tumor rejection when treating with CD8 T cells KO with “combo” vs either “AA” or “AB” constructs. Thus, all the assays to evaluate the impact of PDHB disruption in CD8 T cells, were performed utilizing both gRNA constructs (“AA+AB” combo”) (FIG. 2B).

Mouse Pdhb gRNA construct-Predesigned IDT construct (AA)
Mm.Cas9.PDHB.1.AA

| | | | | On- | Off- |
|---|---|---|---|---|---|
| | | | | target | target |
| Position | Strand | Target Sequence | PAM | score | score |
| 8171504 | −— | GTTCGTGAAGCTATTAATCA | AGG | 64 | 67 |
| | | (SEQ ID NO: 3) | | | |

gRNA Sequence
GUUCGUGAAGCUAUUAAUCAGUUUUAGAGCUAUGCU (SEQ ID NO: 4)
ACCUGGGUUAUUAUCACGAAGUUUUAGAGCUAUGCU (SEQ ID NO: 5)

Human IDT gRNA constructs to target Pdhb.
Hs.Cas9.PDHB.1.AA

| | | | | On- | Off- |
|---|---|---|---|---|---|
| | | | | target | target |
| Position | Strand | Target Sequence | PAM | score | score |
| 58430844 | − | GCCAAGACCTACTACATGTC | TGG | 59 | 74 |
| | | (SEQ ID NO: 1) | | | |

Hs.Cas9.PDHB. 1.AB

| | | | | On- | Off- |
|---|---|---|---|---|---|
| | | | | target | target |
| Position | Strand | Target Sequence | PAM | score | score |
| 58430715 | + | GGACTGACCACCTTTAAGCC | TGG | 67 | 70 |
| | | (SEQ ID NO: 2) | | | |

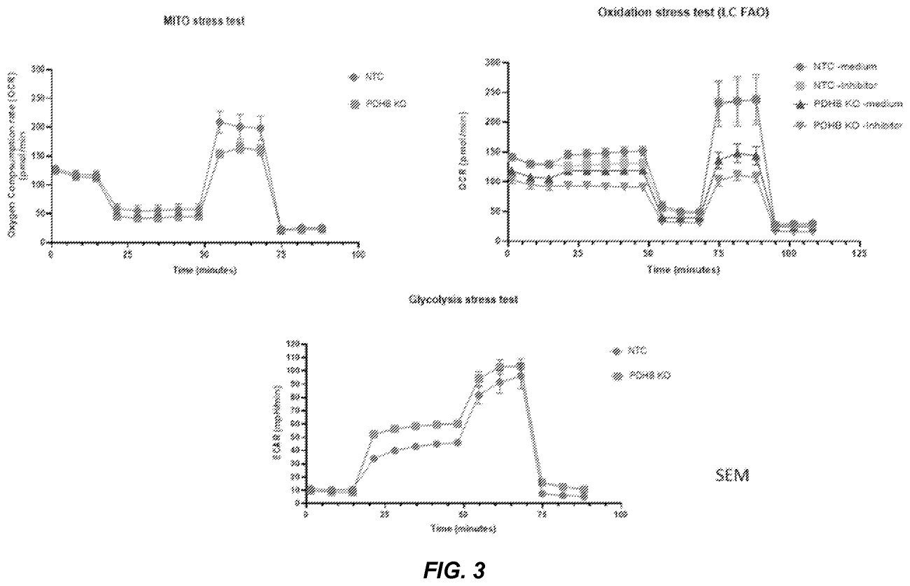

Due to the essential role of PDHB as a linker between glycolysis and TCA cycle, we performed some functional metabolic assays (seahorse assays) to evaluate mitochondrial function (Mito Stress Test and Fatty acid β-oxidation, FAO) and measure the activity of the glycolytic pathway after glucose starvation (Glycolysis Stress Test). The studies showed that PDHB CD8 T cells have an impaired mitochondrial function with reduced respiratory capacity and FAO capacity while having an increased glycolytic activity (FIG. 3).

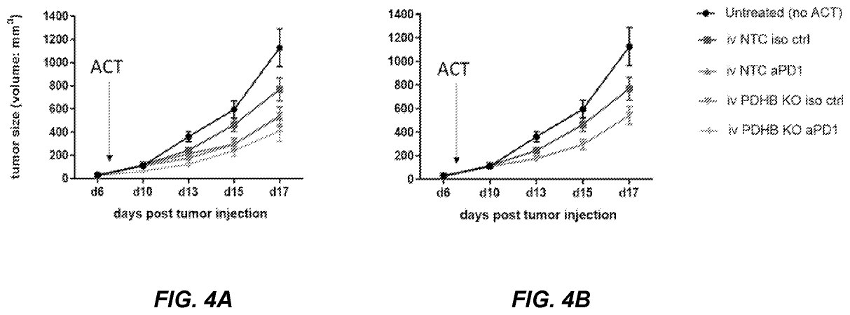

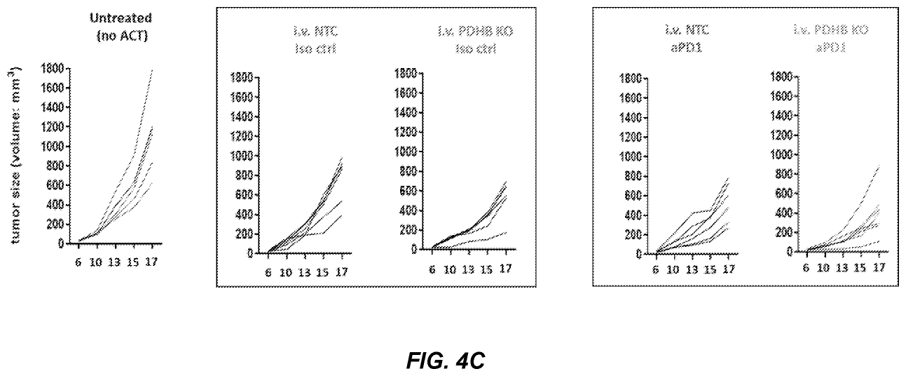

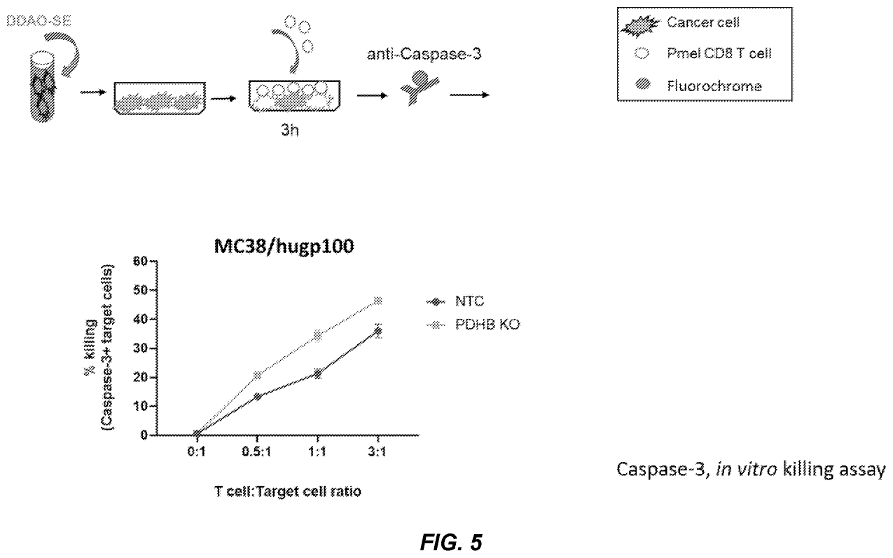

ACT studies in a preclinical melanoma model (B16) (FIG. 4C), along with our in vitro killing (FIG. 5) and IFNg expression assays (FIG. 6), suggest that PDHB disruption improves tumor rejection.

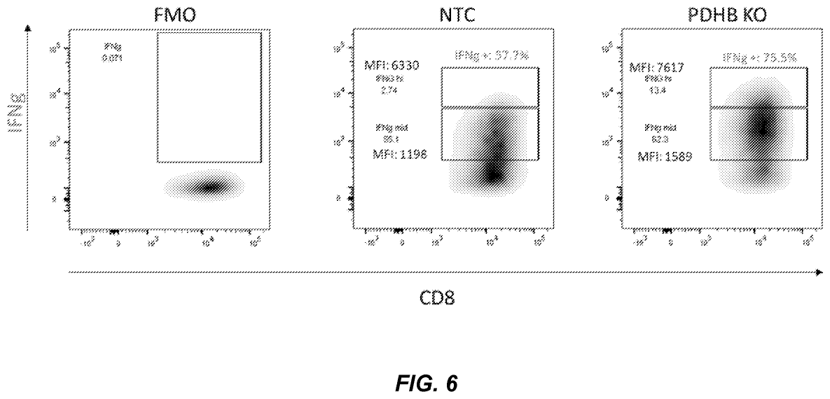

To understand the metabolic changes and underlaying mechanisms that may induce the stronger tumoricidal capacity in the PDHB KO T cells, mRNAseq and untargeted metabolomics studies were performed (FIG. 7, a schematic representation of the sample preparation for mRNAseq or metabolomics).

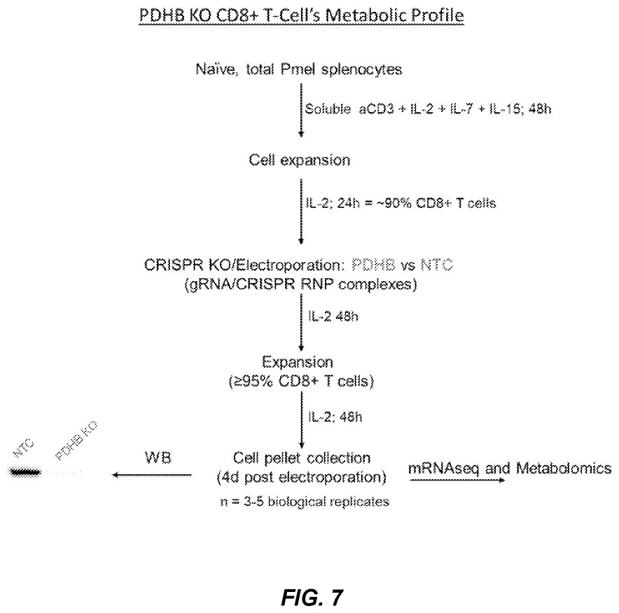

Transcription and pathway analysis indicated that PDHB genetic disruption, strongly induces lipid biosynthesis (FIG. 8).

The top 15 most significantly upregulated pathways (mainly related to lipid biosynthesis) were superpathway of cholesterol biosynthesis, cholesterol biosynthesis I, cholesterol biosynthesis II (via 24,25-dihydrolanosterol), cholesterol biosynthesis III (via desmosterol), role of hypercytokinemia/hyperchemokinemia in the pathogenesis of influenza, mevalonate pathway I (isoprenoid pathway), acetate conversion to acetyl-CoA, superpathway of geranylgeranyldiphosphate biosynthesis I (via mevalonate), VDR/RXR activation, G protein coupled receptor signaling, phagosome formation, ethanol degradation IV, and white adipose tissue browning pathway.

The metabolomics analysis focused on the most upregulated metabolites in the PDHB KO cells revealed that inosine was within the top 5 upregulated metabolites (Table 1). Since glucose is highly depleted by cancer cells in the TME, and inosine can serve as an alternative carbon source for CD8 T cells the upregulation of inosine in the PDHB KO T cells might support T-cell proliferation and function in the TME, in line with our observations in the preclinical melanoma studies.

In addition, purines and pyrimidines were among the most significantly upregulated metabolites.

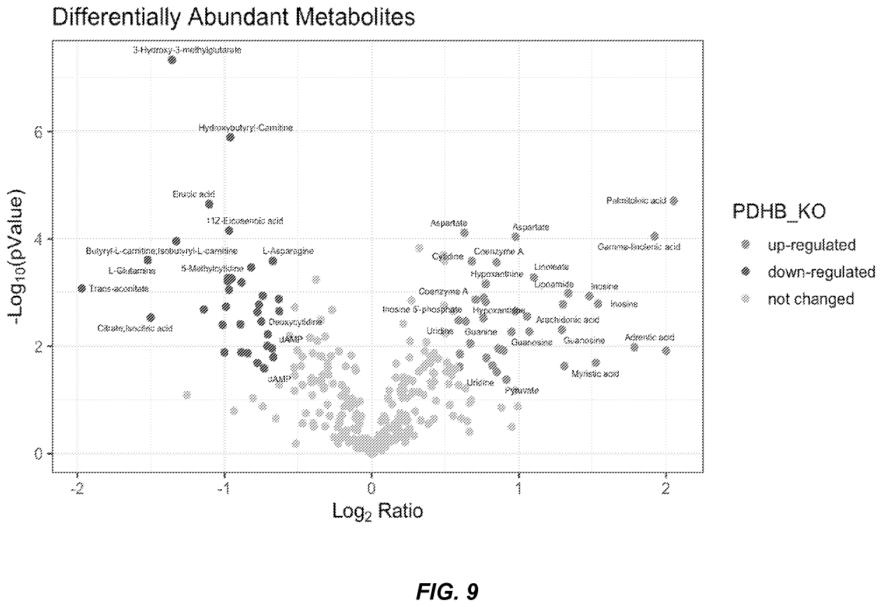

The integrated transcriptome, metabolomics and pathway analyses indicated that the elimination of PDHB in T cells, induces a metabolic reprograming characterized by the upregulation of unsaturated fatty acid biosynthesis, glycolysis/gluconeogenesis, pyruvate metabolism, chemokine signaling and purine metabolism (FIG. 10).

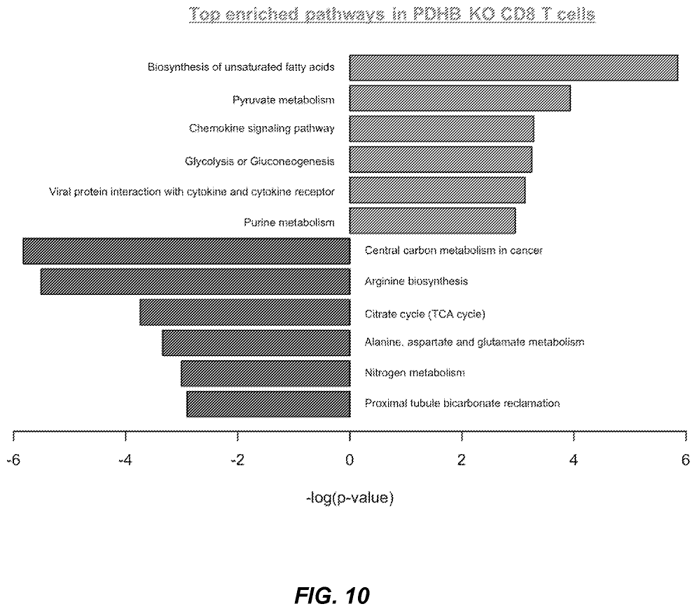

Remarkably, the transcriptome analysis suggests that the PDHB KO CD8 T cells have an expression pattern of Tscm/Tcm cells (FIG. 11) which might be associated with the capacity of the KO cells to infiltrate and survive within tumors. Further phenotypic analyses are required to determine the protein expression of those molecules.

Overall, these data suggests that the efficient disruption (KO) of PDHB in T-cells could contribute to the improvement of the T-cell adoptive therapy.

In conclusion, these data indicate that the metabolomic reprograming of T cells by the strategic disruption of a metabolic gene with CRISPR/Cas9 RNP could serve as a powerful approach to generate engineered T cells able to overcome one of the major barriers imparted by tumors on effector T cells, the metabolic restriction and toxicity in the TME.

TABLE 1
Top-5 most differentially abundant metabolites

| Row.Identity..all.IDs. | NTC_mean | KO_mean | log2_ratio | Welch.T.test | Significance |
|---|---|---|---|---|---|
| Palmitoleic acid | 21.59052 | 23.64308 | 2.052557 | 1.96E−05 | p-value < 0.05 |
| 2-Hydroxyhexadecanoic acid; | 17.1152 | 19.11494 | 1.999739 | 0.012044 | p-value < 0.05 |
| 3-Hydroxyhexadecanoic acid; | | | | | |
| 16-Hydroxyhexadecanoic acid | | | | | |
| Gamma-linolenic acid | 16.36329 | 18.284 | 1.92071 | 8.96E−05 | p-value < 0.05 |
| Adrentic acid | 17.56678 | 19.35143 | 1.784652 | 0.01046 | p-value < 0.05 |
| Inosine | 22.67911 | 24.21503 | 1.535917 | 0.001609 | p-value < 0.05 |

### Example 2. Electroporation of Pmek CD8 T Cells—100 ul Neon System

Sample: Pmek CD8 T cells 48 h activation aCD3+ CKs, followed by 24 h expansion with IL2.

### Before Electroporation Day, Check Reagent, Label Vials, and Split T Cells

Reconstitute new crRNAs and tracrRNA (they withstand freeze/thaw cycles, no need to aliquot once reconstituted. Work inside hood). For all reagents, to reconstitute, spin down vials for few seconds. After IDT buffer addition into the bottom of the vial, incubate at RT for 20 min before using or freezing. Store @ −20° C.

IDT crRNA comes as 2 or 10 nmol solid, reconstitute at 200 uM to use. Add 10 ul for 2 nmol reagent (or 50 ul for 10 nmol reagent) of IDTE buffer directly into the bottom and mix by pipetting up and down.

IDT tracrRNA (ATTO_550-tagged) (Cat #1075928) comes as 20 nmol solid reagent, reconstitute at 200 uM, add 100 uL IDTE buffer directly into the bottom, and mix by pipetting up and down, protecting from light.

IDT electroporation enhancer =comes as 10 nmol solid reagent, reconstitute at 100 uM, add 100 uL IDTE buffer.

Label 2 sets of sterile PCR vials based on table 1, 3; 1 set 1.5 ml Eppendorf vials. Split cells, adjusting to 0.4 M/ml with IL-2 @ 500 IU/ml

### Electroporation

### Set up:

Count T cells (after cell count, keep cells in their old media at 37 C until electroporation)
Spin down all reagents (Cas9 protein, crRNA-NTC, tracrRNA-ATTO_550), keep on ice until electroporation
E2 buffer (ready to use, 4C) (100 ul system)
IDTE buffer (Cat #Nov. 1, 2002-02, ready to use, 4C)
T buffer (ready to use, 4C)
Electroporation 100 ul tips and Electroporation tubes
Set up (2) 6-well plates: Plate 1A→3 wells=PDHB KO. 1B Plate→3 wells NTC+1 well No RNP. Add 2.4 ml/well 20% FBS T cell media antibiotics free+IL-2 and pre-warm it in the incubator (for final cell concentration 0.4×10\hat{ }6 cells/ml=1×10\hat{ }6/2.5 ml final volume).
Label 7 wells in of a 96-U well plate+100 ul/well 20% FBS T cell media antibiotics free+IL-2 and pre-warm it in the incubator (to test cell aliquots for electroporation efficiency based on ATTO_550+ cells)
   Prepare the crRNA+tracrRNA Duplexes. (Sterile PCR Vials, a Vial per Construct per Sample)
(Inside hood, turn off light to protect the tracrRNA reagent).—Follow the 50% 50% rule (equal volumes of reagents)

TABLE 2

| | | | Volume | Volume | |
|---|---|---|---|---|---|
| | Volume | Volume | PDBH | PDHB | |
| | (1) | NTC | KO AA | KO AB | Concentration |
| IDTE Buffer | 2.8 ul | 8.12 ul | 5.18 ul | 5.18 ul | |
| (pH 7.5)* | | | | | |
| cRNA | 1.1 ul | 3.19 ul | 2.04 ul | 2.04 ul | 200 uM |
| tracrRNA | 1.1 ul | 3.19 ul | 2.04 ul | 2.04 ul | 200 uM |
| total volume | 5.0 ul | 14.5 ul | 9.26 ul | 9.26 ul | 44 uM final |
| | | | | | conc. of each |

Mix crRNA, tracrRNA and butter by pipetting up/down, protect trom light, and spin down.
1. NTC
2. PDHB KO AA
3. PDHB KO AB
Spin down, incubate at 95° C. for 5 min in thermocycler (program: “95° C. for 5 min”). Spin down
Allow to cool to room temperature on the benchtop protected from light-at least 10-15 min: if you mix in Cas9 while still hot, the enzyme will degrade!!!

### Prepare Diluted Cas9 Nuclease (Sterile 0.5 ml Eppendorf Vial): Cas9/T Buffer=0.66

Dilute IDT Alt-R S.p. Cas9 nuclease (Cat #1081059)—comes as solution (at −20 C); it is a bubbling solution, spin/down and carefully mix by pipetting up/down. It can withstand freeze/thaw cycles→no need to aliquot. Keep on ice. (Inside hood, mix cas9 reagent very slowly up/down a couple of times, prepare dilution in a sterile vial). Mix very slowly by pipetting up/down a couple of times, keep diluted Cas9 on ice.

TABLE 3

| | Volume (ul) | Volume (ul) |
|---|---|---|
| | (1 sample) | For multiple samples |
| T buffer | 1.5 | 17.4 (T buffer!!!) |
| Alt-R S.p. Cas9 Nuclease | 1 | 11.6 |
| 3NLS (61 uM) | | |
| total volume | 2.5 | 29 |
| final concentration | 24.4 uM | 24.4 uM |

Combine the crRNA:tracrRNA Duplex and Cas9 Nuclease (=RNP)
(Inside hood, turn off light to protect the tracrRNA reagent). Follow the 50% 50% rule (equal volumes of reagents). Prepare the reaction in 0.5 ml Eppendorf vials. Keep on ice

TABLE 4

| | | PDHB | | PDHB | |
|---|---|---|---|---|---|
| | NTC | KO AA | | KO AB | |
| crRNA # NTC: tracrRNA duplex | 12 ul | 6.75 | ul | 6.75 | ul |
| Cas9 dilution | 12 ul | 6.75 | ul | 6.75 | ul |
| total volume | 24 ul | 13.5 | ul | 13.5 | ul |

### Samples: (#Vial per Construct per Sample)

NTC
PDHB KO AA
PDHB KO AB
Mix very slowly up/down a couple of times, and spin down
Incubate the mixture at RT° 20 min protected from light. After 20 min, transfer to ice or 4° C. (protected from light)

### In the Meantime:

Prepare the electroporation enhancer reagent: make 22 uM solution (equal to duplex): 36.6 uL IDTE buffer+10.4 ul enhancer, final vl=47 ul
Set up the Neon device: put the pipette station in the hood, plug in the machine, turn it on and select the protocol: 1600V, 10 ms, and 3 pulses. Fill a provided tube with 3 ml E2 buffer for (each 3 ml buffer/electroporation tube can be used up to 10× with different samples) (Electroporation tips can be used up to 2× if same sample). Insert the Neon tube with the E2 buffer into the electroporator station
Prepare T cells in T buffer: Wash cells twice with PBS (w.o. Ca2+ and Mg2+; FBS contains RNases and DNases that will reduce efficiency; 1500 rpm for 5 minutes (1M/well), adjust cells to 11.3M cells/ml T buffer immediately before electroporation. Keep on ice. Note: The T and E2 buffers are toxic to the cells. Do not leave the cells in the buffer for >10 min.
Tip: keep cells in PBS before the second spin down, and only when fully prepared for the electroporation, do the second spin down, keeping the sample on ice, and only remove PBS to add the T cell buffer inside the hood when ready to add T cells/T buffer to the RNP complexes.
Prepare T cells→9M/793 ul T buffer needed→Prepare for ˜2×⇒18 M/1,586 ul T buffer. Take an aliquot for cell count once cells resuspended in 2^{nd} PBS to confirm cell #, and continue with last wash.

### Electroporation (Only Process 5 Samples per Batch of T Cell Suspension)

Combine the RNP+electroporation enhancer+T cells: USE 1.5 ml EPPENDORF VIALS
Add to the RNP, electroporation enhancer, and then add the respective T cell volume. Work per vial.
Place RNP+Enhancer mixture on ice protected from light until T cells are ready to be added.

### *PMEL Plates (1A, 1B)

Plate 1A: PDHB KO combo AA+AB (3 wells)
Plate 1B: NTC (3 wells)+Electroporated no RNP (1 well)

TABLE 5

| | | | | PDHB KO Combo | |
|---|---|---|---|---|---|
| | Single construct | NTC | | (AA + AB) | |
| | and 1 well | 3 wells | | 3 wells | |
| Diluted | 7 ul | 21 | ul | 21 | ul |
| Electroporation | | | | | |
| enhancer | | | | | |
| RNP | 7 ul | 21 | ul | 10.5 ul AA + | |
| | | | | 10.5 ul AB | |
| T cell | 113 ul (~1.28M | 339 | ul | 339 | ul |
| suspension | cells) | | | | |
| Total volume | 127 ul (take 100 | 381 | ul | 381 | ul |
| | ul** Neon tip) | | | | |

$$Electroporated no construct ( 1 well ) = 14 ul PBS + 113 ul cells$$

With the Neon pipette, grab a Neon tip by pushing the head of the pipette fully down on (an “spider” arm comes out), push it down onto the Neon tip to grab it, making sure the tip is well attached, and aspirate very slowly 100 ul sample (RNP+enhancer+Tcells mix in buffer). Do not pipette up and down, it is very important to avoid air bubbles in the Neon pipette tip; if there is an air bubble, discard carefully that sample, and aspirate again the cell suspension very slowly!
Insert the Neon tip+pipette with sample into the cuvette+buffer set up in the electroporation station: you will hear a click when the pipette is well inserted into the cuvette. Press “start” on the Neon screen, you will hear 1+2 beeps, and wait until the “complete” message is shown to take out the Neon pipette+tip with sample.
Dispense the cell suspension in a medium-containing well (6-well plate). Do not pipette up and down. Gently swirl the plate to distribute cells homogenously. Take a 125 ul-aliquote and transfer to the 96-U well plate. Incubate 6-well plate at 37° C./5.0% CO2 for 24 h
Use the 125 ul aliquot (=50,000 cells) to evaluate electroporation efficiency (ATTO_550 fluorescence) within 3-24 h post electroporation (tracer reaction pick @ 24 h; after 48 h the fluorescence is not present). Incubate until Flow acquisition

### Electroporation Efficiency Evaluation

Within 1-24 h post electroporation: Spin down the 96-well plate, discard s/n; resuspend cell pellet in 200 ul FACs, spin down, discard s/n. Add 100 ul diluted Live/Dead dye. Incubate ˜20 min 4 C protected from light. Wash with 125 ul FACS buffer, wash again with 200 ul FACS buffer. Resuspend cell pellet in 200 ul FACS buffer, transfer to FACs tubes, add 150 ul FACS for 350 ul final volume. Store @ 4° C. protected from light until acquisition. Evaluate ATTO_550 expression by FC (acquire ˜3K events, from live gate)

### *Prepare Beads:

$$Compbeads = unstained , PE ( 0.3 ul any PE Ab )$$

$$LD beads = 0.3 ul LD Aqua dye$$

Refresh media without Antibiotics, by 24 h post electroporation
Add fresh media without antibiotics+IL-2: Without disturbing cells (do not mix, add media by slowly placing the tip against the wall of the well), add 2.5 ml of fresh 20% FBS T cell media without antibiotics+IL-2/well. Calculate IL-2 based on the volume of the freshly prepare media.
Incubate @ 37 C/5.0% CO2, 24 h.

### Cell Expansion:

Expand cells 48 h post electroporation: Leave as 0.4 M/mL with IL-2 in complete T cell media (10% FBS with antibiotics).

### Cell Expansion Am):

Expand cells: Leave as 0.4 M/mL with IL-2 in complete T cell media (10% FBS with antibiotics).
Functional assay (d4 post electroporation)
Functional assay—see separate protocol
Collect 1 vial 5M cells (WB) and 1-2 vials 10M cells (omics)

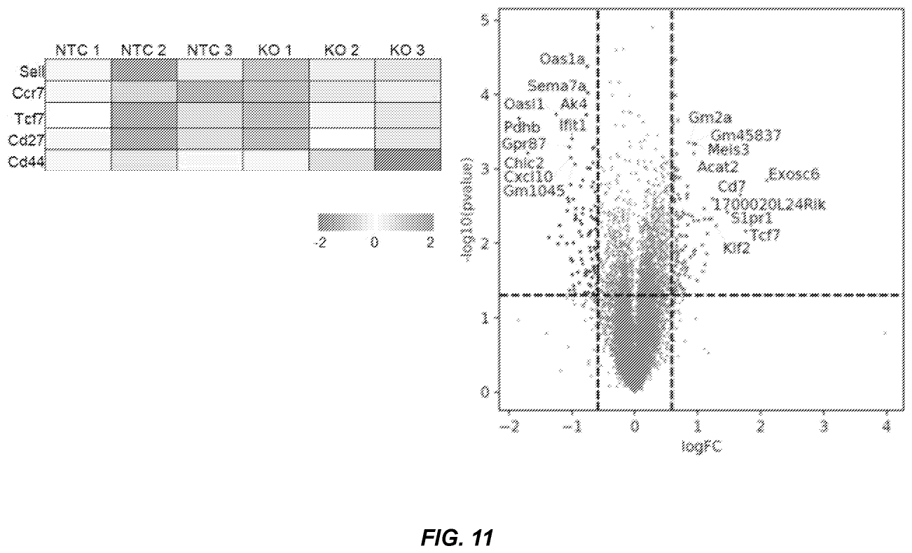

FIG. 12 a multiplex CRISPR/Cas9 RNP method to simultaneously target up to three genes. For the multiplex RNP targeting, each gene can be disrupted using an individual RPN complex that carries out a specific gRNA construct for the GOI. Thus, RPN 1 targets GOI 1, RNP 2 targets GOI 2, and RNP 3 targets GOI 3. IDT shared two fluorescent tracers not commercially available yet, to track each RNP complexes. The tracer that forms part of each RNP complex is tracked by the expression of either ATTO_488, ATT_647 or ATTO_550. The three surface molecules targeted by the multiplex RNP method: VISTA, PD-1 and LAG-3.

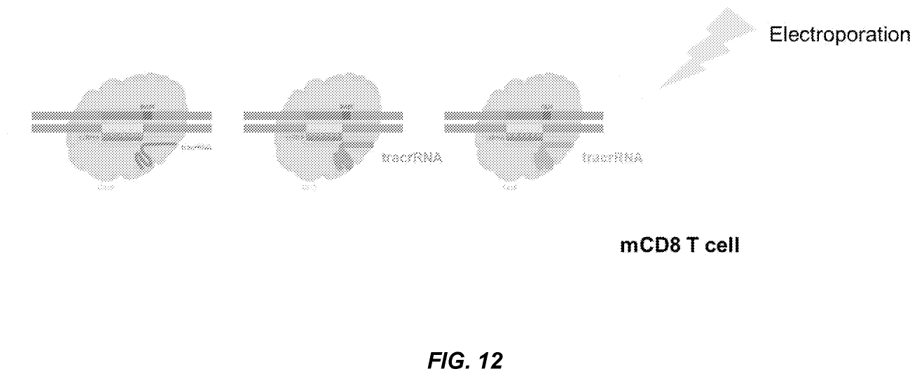

FIG. 13A is a flow chart indicating the sample preparation to perform KO. Different conditions were evaluated: single KO (single RNP for each of the target molecules VISTA, PD1 and LAG-3), double KO (combination of two different types of RNPs targeting either VISTA and PD1, or VISTA and LAG3), triple KO combination of all RNPs to simultaneously target VISTA, PD1 and LAG3 withing the same T-cell suspension. FIG. 13B shows electroporation efficiency determined within 24 h post electroporation by FC. Live T cells were gated and the presence of each of the constructs (RNPs) in CD8 T cells was tracked by the expression of ATTO_550, ATTO_488 and ATT_647. Each tracer reflects the presence of each type of RPN used to target different genes/molecules (VISTA, PD-1 and LAG-3).

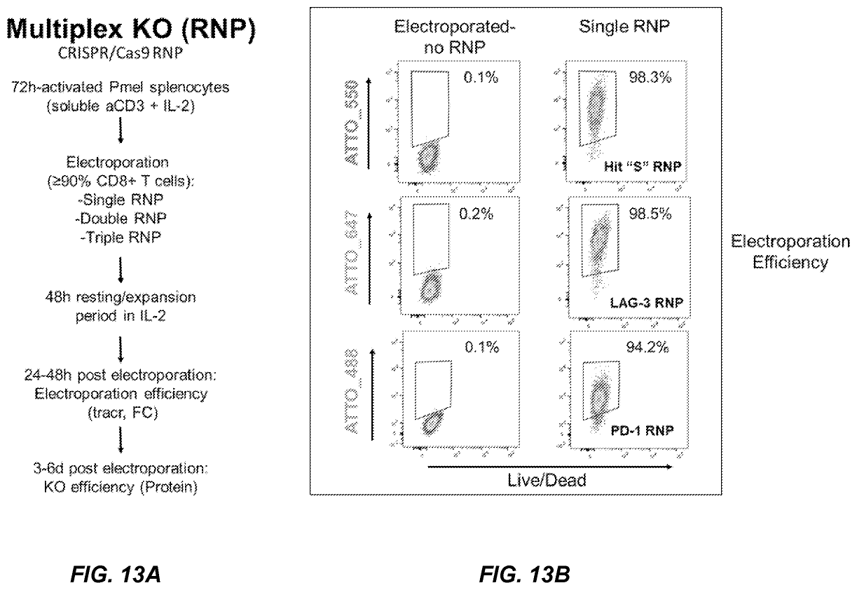

FIG. 14 shows evaluation of KO levels (%) induced in CD8 T cells by each RPN construct targeting either VISTA (left plots), PD1 (middle plots) or LAG3 (right plots). Upper panel. Basal levels of marker expression in NTC T cells (% in black #s). Lower panel, the KO levels were determined by comparison to the expression of each maker in CD8 T cells transduced with NTC (percentage of KO efficacy in red, % KO; remaining protein expression in black % #). Flow cytometry done 4 days post electroporation. Live T cells were gated based on CD8 expression. This data indicates the maximum KO level that each RNP can achieve for the specific targets.

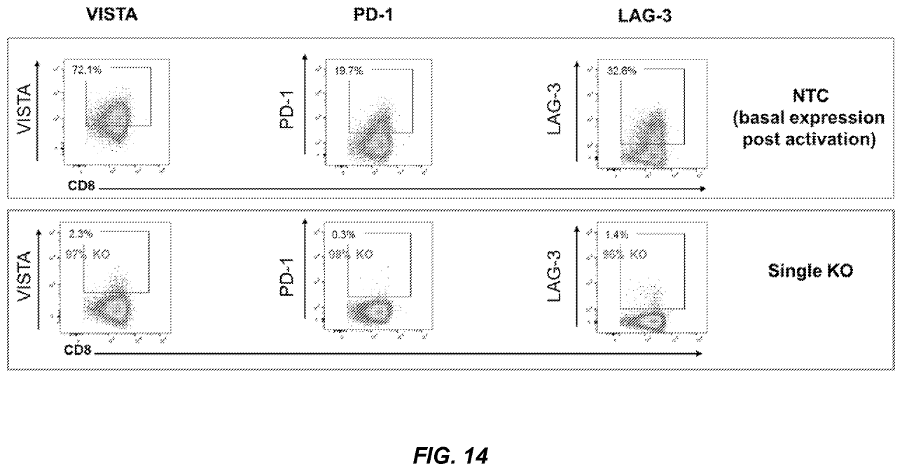

FIG. 15 shows evaluation of KO levels (%) induced in CD8 T cells when targeting two molecules simultaneously. Two RPNs constructs targeting either VISTA and PD1 (upper panel) or VISTA and LAG3 (lower panel) were utilized in the reaction. The KO levels were determined by comparison to the level of each maker in CD8 T cells transduced with NTC (basal levels indicated in previous slide, upper panel). The percentage of KO efficacy indicated in red, % KO; remaining protein expression in black % # (upper and lower panels). Flow cytometry done 4 days post electroporation. Live T cells were gated based on CD8 expression. This data indicates the maximum KO level that is achieved when two RNPs targeting two genes are used in a single reaction.

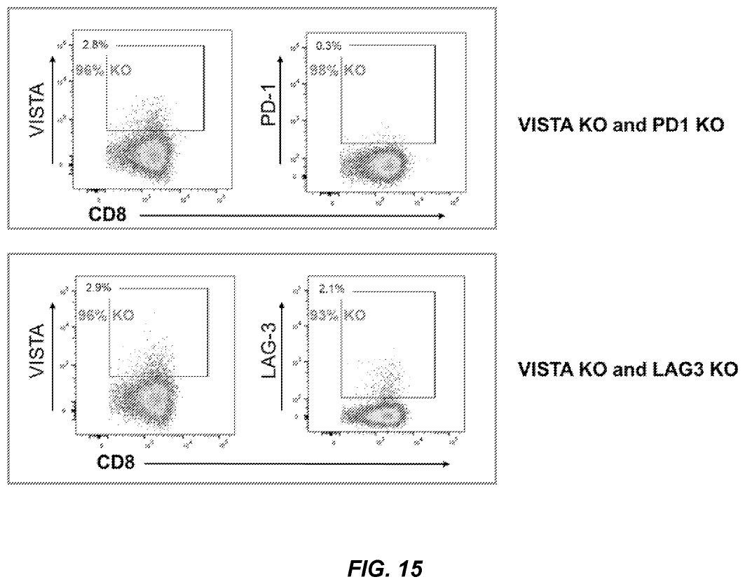

FIG. 16 shows evaluation of KO levels (%) induced in CD8 T cells when targeting three molecules simultaneously. Three RPNs constructs targeting VISTA, PD1 and LAG3 were utilized in the reaction. The KO levels were determined by comparison to the level of each maker in CD8 T cells transduced with NTC (basal values indicated in slide 18, upper panel). The percentage of KO efficacy indicated in red, % KO; remaining protein expression in black % #. Flow cytometry done 4 days post electroporation. Live T cells were gated based on CD8 expression. This data indicates the maximum KO level that is achieved when simultaneously using three RNPs to target three genes in a single reaction.

### Example 3

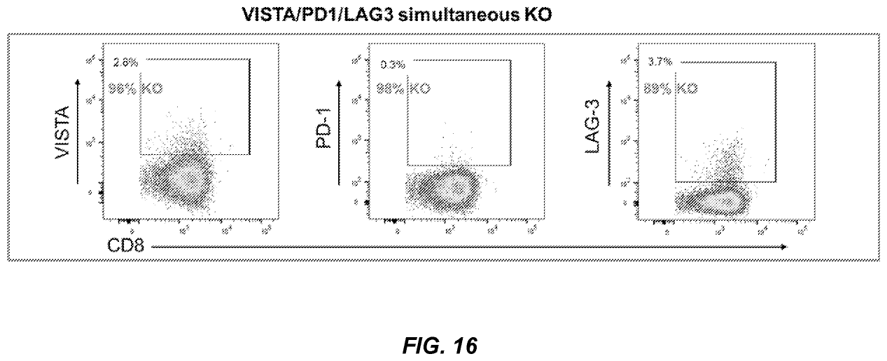

FIG. 17 illustrates the PDH complex.

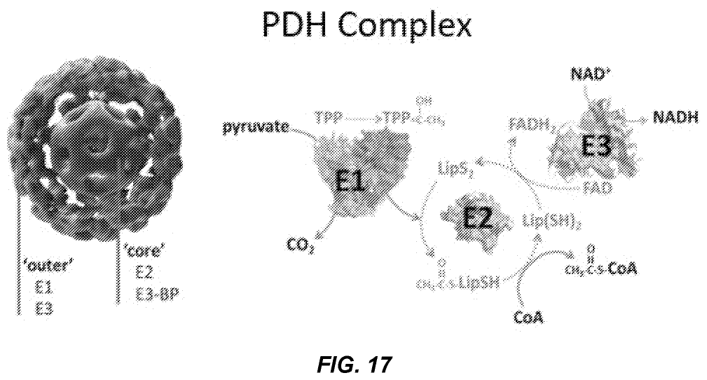

FIG. 18 illustrates the role of the PHD complex in the metabolic pathway.

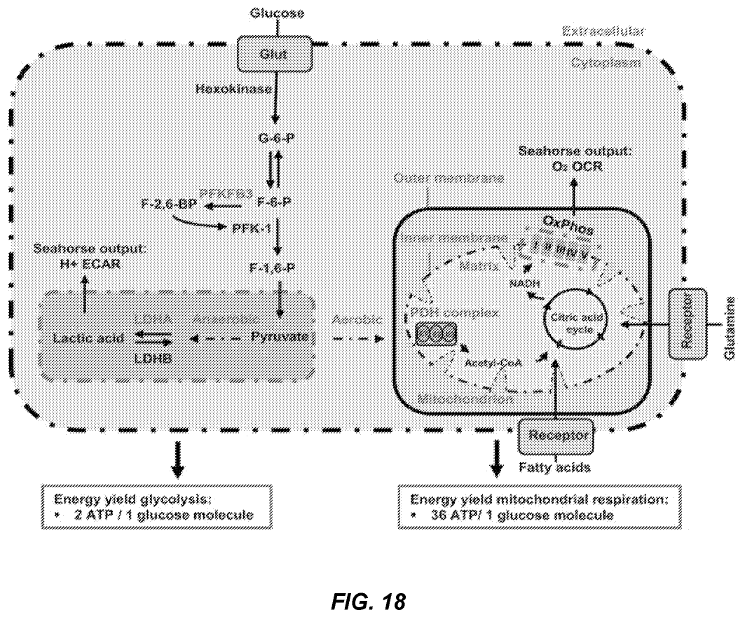

FIG. 19 illustrates a scheme used for T cell activation, expiation, CRISPR RNP transfection, PDHB KO evaluation, and validation.

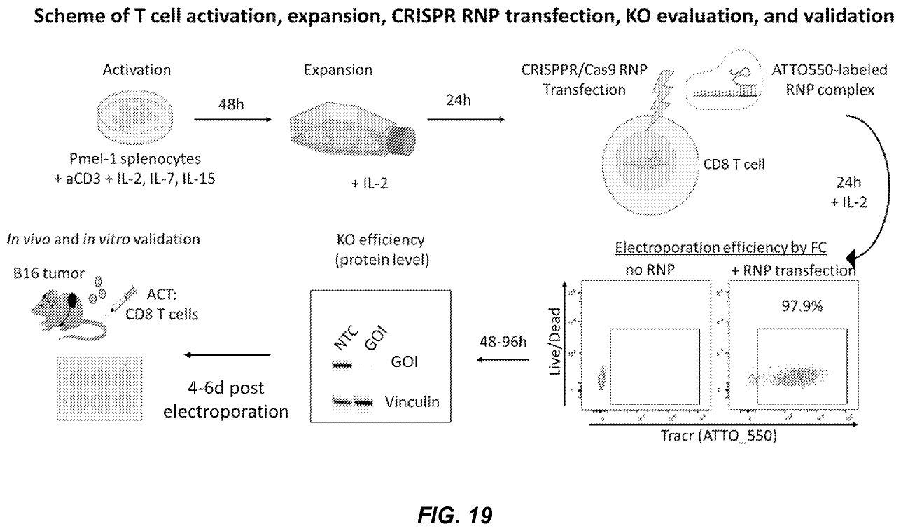

FIGS. 20A to 2D show disruption of the Metabolic Hit PDHB Enhances Tumor Infiltration of Adoptively Transferred CD8 T Cells.

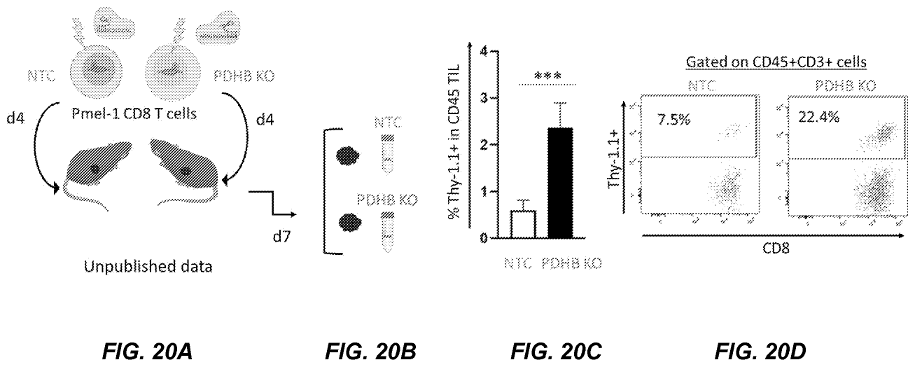

FIGS. 21A to 21C show mitochondrial stress test (FIG. 21), oxidative stress test (FIG. 21B), and glycolysis stress test (FIG. 21C) results for NTC and PDHB KO T cells.

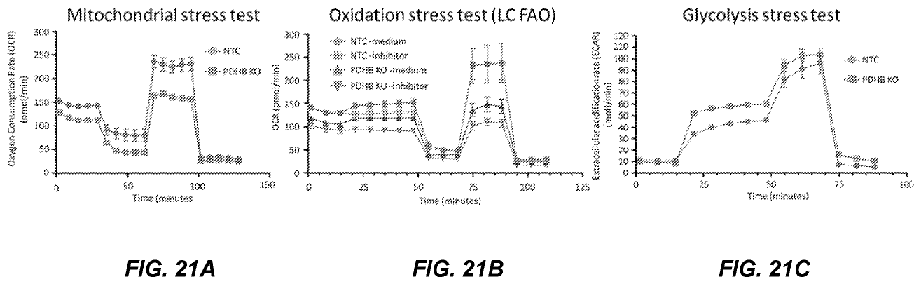

FIGS. 22A and 20B show PDHB KO CD8 T cells display enhanced anti-tumor activity in Adoptive Cell Therapy (ACT) by intravenous (FIG. 22A) and intratumor (FIG. 22B) delivery.

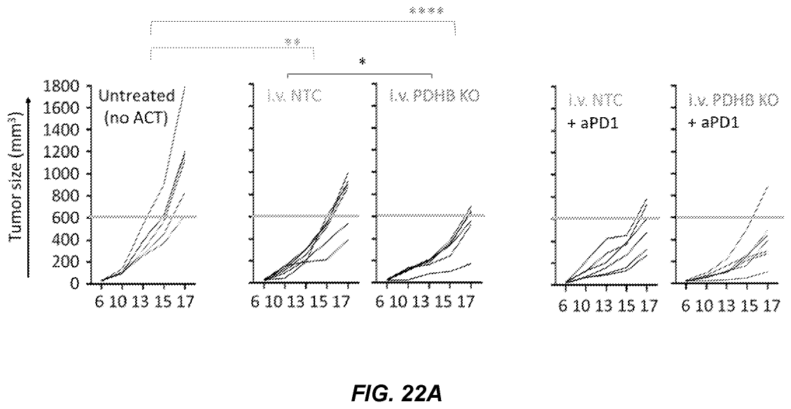

FIGS. 23A and 23B show PDHB KO CD8 T cells display higher killing potential (FIG. 23A) and IFN-γ production (FIG. 23B).

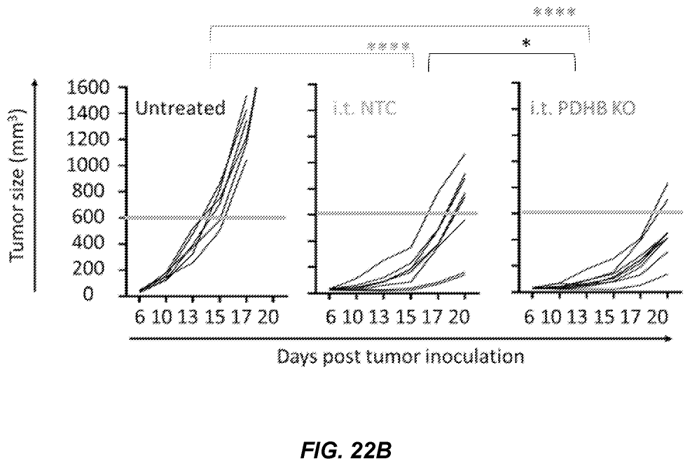

FIG. 24 shows untargeted metabolomic profiling identified distinct metabolic reprograming in PDHB KO CD8 T cells.

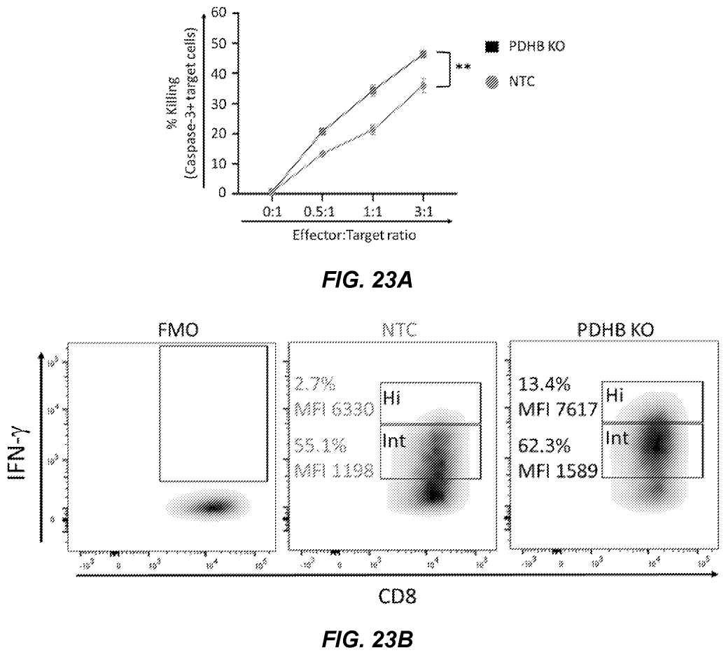

FIG. 25 shows untargeted metabolomics revealed metabolic changes associated to the purine synthesis pathway. Boxes are upregulated metabolites in PDHB KO CD8 T cells. Inosine is Highly Expressed in PDHB KO CD8 T Cells (Table 6.)

TABLE 6

| | NTC_mean | KO_mean | Log2_ratio | Welch.T.test | Significance |
|---|---|---|---|---|---|
| Palmitoleic acid | 21.59052 | 23.64308 | 2.052557 | 1.96E−05 | p-value < 0.05 |
| Palmitic acid | 17.1152 | 19.11494 | 1.999739 | 0.012044 | p-value < 0.05 |
| Gamma Linoleic acid | 16.36329 | 18.284 | 1.92071 | 8.96E−05 | p-value < 0.05 |
| Adrenic acid | 17.56678 | 19.35143 | 1.784652 | 0.01046 | p-value < 0.05 |
| Inosine | 22.6911 | 24.21503 | 1.535917 | 0.001609 | p-value < 0.05 |

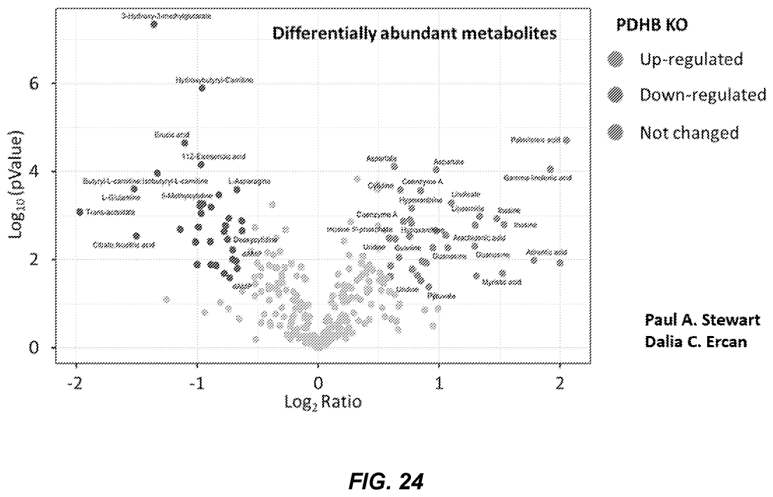

Purine nucleoside phosphorylase (PNP) catalyzes inosine to hypoxanthine and Ribose-1-P. As shown in FIG. 26, inosine hydrolysis is required for Increased IFN-γ production in PDHB KO CD8 T Cells.

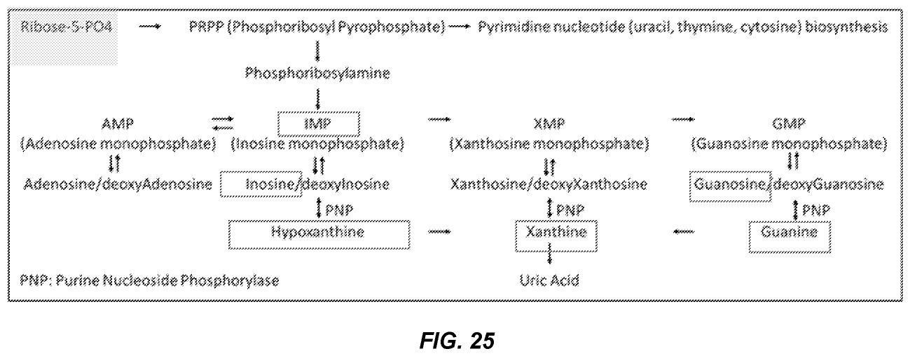

FIG. 27 shows ¹³C6-Glucose tracer analysis in PDHB KO vs NTC CD8 T cells to elucidate the inosine source in PDHB KO CD8 T cells.

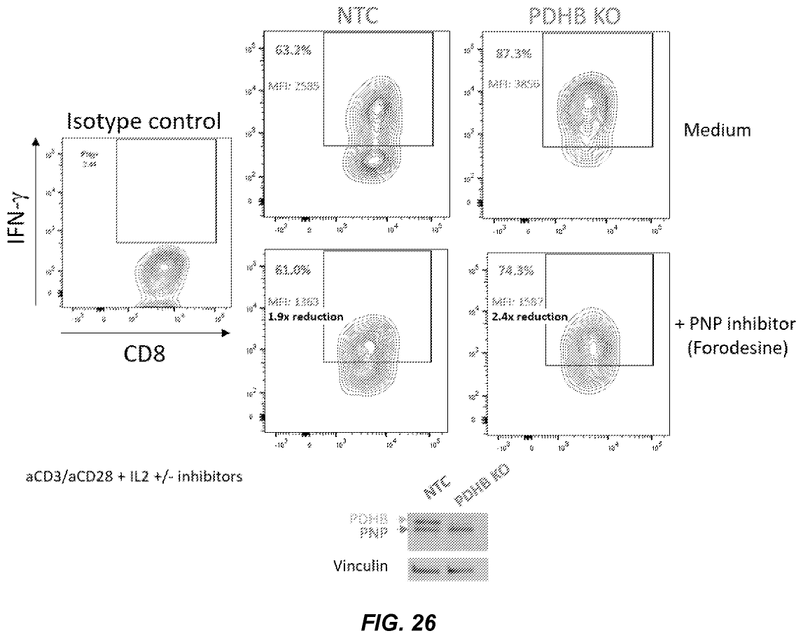

FIG. 28 shows glucose metabolism via the pentose phosphate pathway leads to increased inosine production in PDHB KO CD8 T cells.

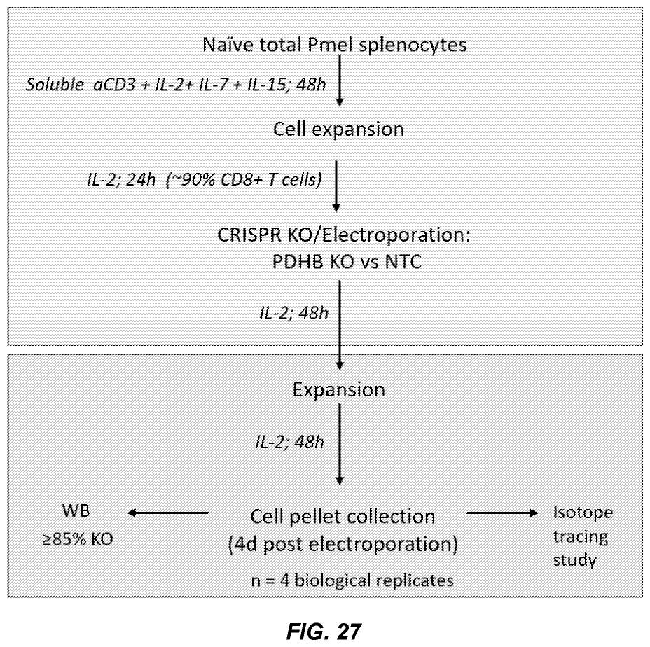

FIG. 29 shows PDHB KO CD8 T cells express higher levels of fatty acids (see Table 6).

Table 7 shows the top enriched pathways in PDHB KO CD8 cells by IPA gene analysis (Qiagen).

TABLE 7
Top enriched pathways in PDHB KO CD8 cells.

| | −log |
|---|---|
| Ingenuity Canonical pathways | (p-value) |
| Superpathway of Cholesterol Biosynthesis | 8.55 |
| Cholesterol Biosynthesis I | 5.54 |
| Cholesterol Biosynthesis II (via 24,25-dihydrolanosterol) | 5.54 |
| Cholesterol Biosynthesis III (via desmosterol) | 5.54 |
| Role of hypercytokinemia/hyperchemokinemia in the | 4.13 |
| pathogenesis of influenza | |
| Mevalonate pathway I | 3.74 |
| Acetate conversion to Acetyl-CoA | 3.41 |
| Superpathway of geranylgeranyldiphosphate biosynthesis I | 3.4 |
| (via melavonate) | |
| VDR/RXR activation | 3.35 |
| G-protein coupled receptor signaling | 3.34 |

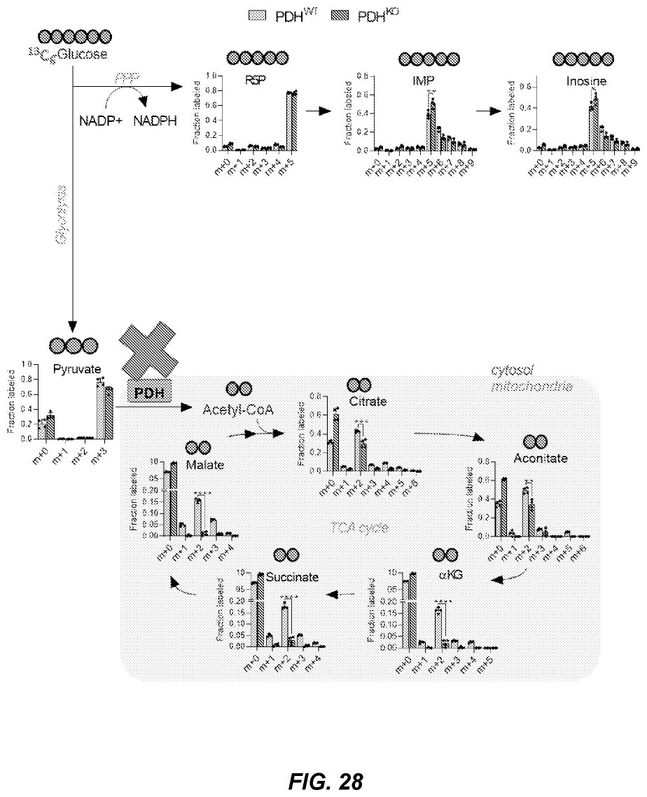

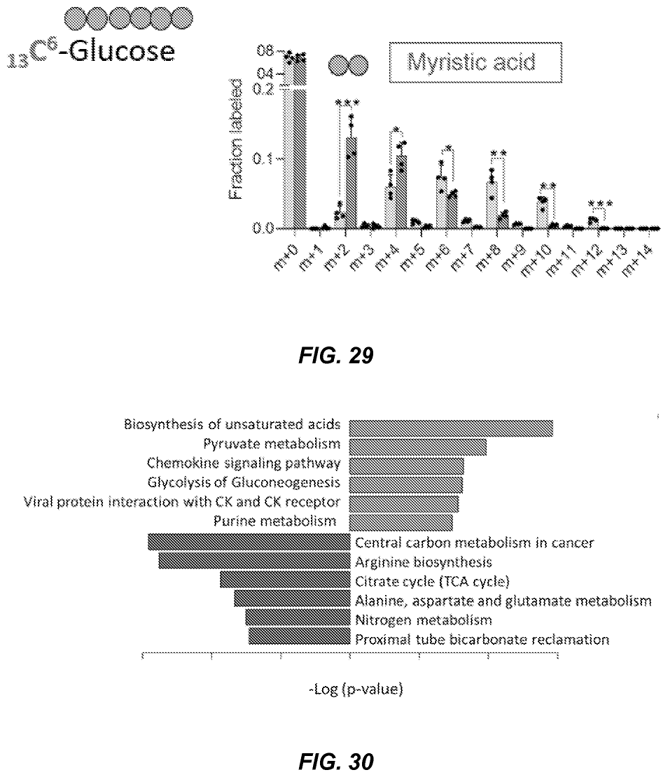

FIG. 30 shows integrated mRNA and metabolomics analysis. FIG. 31 illustrates a potential model of PDH bypass via Acetyl-CoA synthetase (ACSS2), which is upregulated in PDHB KO T cells.

FIG. 31 illustrates a potential model of PDH bypass via Acetyl-CoA synthetase (ACSS2), which is upregulated in PDHB KO T cells.

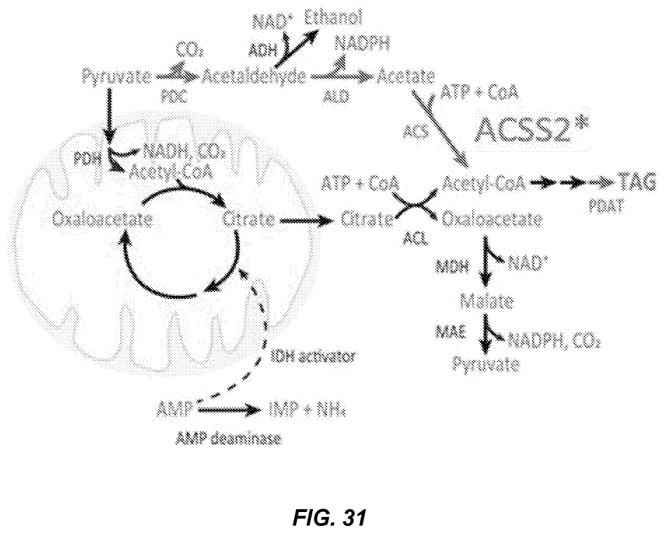

FIG. 32 shows PDHB disruption strongly increases CD8 T Cell proliferation. A B16.F10 spheroid pre-treated with IFNγ was cultured in a liquid-like-solid (LLS) material (packed granular gel) and treated with PDHB KO or WT T cells from D0-D3. Images from left-top, to left-bottom, and then right-top to right-bottom are t=0 h, t=5 h, t=10 h, t=15 h, t=20 h, t=25 h, t=30, h and t=35 h.

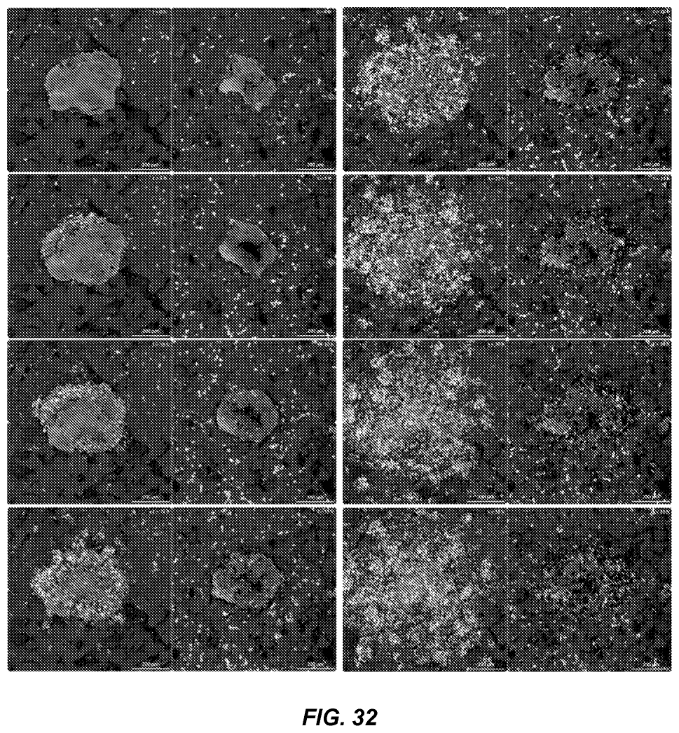

FIG. 33 shows hyperproliferative PDHB KO CD8 T Cells produce higher levels of IFN-γ.

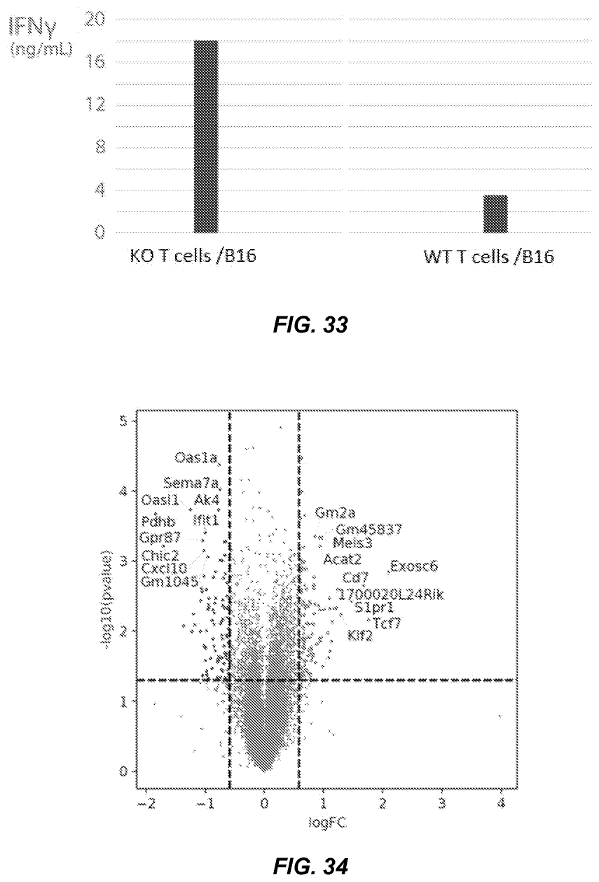

Single-Cell RNA-Seq analysis Revealed distinct effector and CM subsets in PDHB KO CD8 T cells, including Gpr141⁺Tnfrsf9⁺ EFF and Ptpn18⁺Stat1⁺Ltb⁺Tcf7⁺ CM cells. PDHB KO CD8 T cells contained a gene expression pattern of Tscm/Tcm cells (FIG. 34).

Single-Cell RNA-Seq Analysis also identified targetable markers to potentiate PDHB KO T cells' antitumor response and enhance ACT, including 4-1BB, LAG-3 and TIM-3.

Table 8 below shows single-Cell RNA-sequencing delineated ex vivo signatures of PDHB KO CD8 T adoptively transferred in B16 tumors. this table shows ex-vivo signatures. In addition, the analysis indicates that the main cell clusters induced in PDHB KO T CD8 T cells vs NTC in the tumor microenvironment are: Cluster 5 (Proliferation), Cluster 1 (Activation), Cluster 0 (Cytotoxic). These three clusters were commonly upregulated in both KO samples vs NTC. Upregulation based on the normalization (based on total cell #, ratio 1 was set for NTC, and from there, ratios calculated for each KO sample).

TABLE 8

| | Sample (Cell #) | | | Ratio | | |
|---|---|---|---|---|---|---|
| Cluster name | 3-NTC | 4-PDHB KO | 6-PDHB KO | 3-NTC | 4. PDHB KO | 6-PDHB KO |
| 0. Cytotoxic | 118 | 367 | 1538 | 1 | 3.1 | 13.0 |
| 13. EFF | 106 | 171 | 54 | 1 | 1.6 | 0.5 |
| 14. EFF | 74 | 98 | 103 | 1 | 1.3 | 1.4 |
| 1. Activation | 88 | 345 | 1422 | 1 | 3.9 | 16.2 |
| 2. CM | 439 | 749 | 544 | 1 | 1.7 | 1.2 |
| 3. Activation | 212 | 696 | 425 | 1 | 2.8 | 2.0 |
| 5. Proliferation | 84 | 371 | 1531 | 1 | 4.4 | 18.2 |
| 6. Activation | 117 | 381 | 569 | 1 | 3.3 | 4.9 |
| 9. EFF | 159 | 345 | 231 | 1 | 2.2 | 1.5 |
| Cell # | 1408 | 3458 | 6510 | | | |
| Ratio | 1 | 2.5 | 4.6 | | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method comprising
(a) collecting immune effector cell from a subject with cancer;
(b) treating the immune effector cell with a PDHB inhibitor or genetically modifying the immune effector cell to inhibit or ablate PDHB expression.

2. The method of claim 1, wherein the PDHB inhibitor is an siRNA, antisense, or gRNA oligonucleotide.

3. The method cell of claim 1, further comprising treating the immune effector cell with a TIM3 inhibitor or genetically modifying the immune effector cell to inhibit or ablate TIM3 expression.

4. The method cell of claim 1, further comprising treating the immune effector cell with a LAG3 inhibitor or genetically modifying the immune effector cell to inhibit or ablate LAG3 expression.

5. The method cell of claim 1, wherein the immune effector cell is engineered to express a chimeric antigen receptor (CAR) polypeptide.

6. The method cell of claim 1, wherein the immune effector cell is a CD8+ lymphocyte.

7. The method cell of claim 6, wherein the immune effector cell is a tumor infiltrating lymphocyte (TIL).

8. A therapeutic cell produced by the method of claim 1.

9. A method of providing an anti-cancer immunity in a subject, comprising administering to the subject an effective amount of the therapeutic cells of claim 8, thereby providing an anti-tumor immunity in the subject.

10. The method of claim 9, further comprising administering to the subject a checkpoint inhibitor.

11. The method of claim 10, wherein the checkpoint inhibitor comprises an anti-PD-1 antibody, anti-PD-L1 antibody, anti-CTLA-4 antibody, anti-OX40 antibody, or a combination thereof.

12. The method of claim 9, further comprising administering to the subject an effective amount of an anti-41BB antibody.

13. The method of claim 9, further comprising administering to the subject an effective amount of an IL-7 inhibitor.

14. The method of claim 9, further comprising administering to the subject an effective amount of an IFN-γ inhibitor.

15. The method of claim 9, further comprising administering to the subject an effective amount of an IL6 inhibitor.

16. A recombinant immune effector cell genetically modified to ablate PDHB expression and one or more of TIM3 and LAG3 expression.

17-22. (canceled)
